(19) United States
(12) Patent Application Publication
(10) Pub. No.: US 2010/0100163 A1
(43) Pub. Date: Apr. 22, 2010

(54) Miniature biofield vitalizer

(76) Inventor: Richard H. Lee, Laguna Niguel, CA (US)

Correspondence Address: RICHARD H. LEE, 22 WOODHAVEN DRIVE, LAGUNA NIGUEL, CA, 92677, US

(21) Appl. No.: 12/288,418

(22) Filed: Oct. 20, 2008

(51) Int. Cl. A61N 1/00; A61B 19/00; A61N 1/39

(52) U.S. Cl. 607/115; 128/898; 607/1

(57) ABSTRACT

An improved method of vitalizing people or other living things using a time varying interaction of electrical fields, electrons, magnetic fields, air and mental in close proximity to the person to be vitalized. The principal embodiment has long battery life and fits in the pocket.

### CROSS REFERENCE TO RELATED APPLICATIONS:

This device draws from U.S. Pat. No. 6,013,021, Biofield Vitalizer by the same inventor. In addition, it utilizes unpredictable signals as disclosed in three patents also by the same inventor. These are U.S. Pat. No. 6,461,316, “Chaos Therapy Method and Device” U.S. Pat. No. 6,770,042, “Therapeutic Signal Combination” and U.S. Pat. No. 7,419,474 “Non-linear Therapy Signal Synthesizer”.

### BACKGROUND OF THE INVENTION

1. Field of the Invention

The field of this invention is biofield vitalizers such as the invention disclosed in U.S. Pat. No. 6,013,021 by the same inventor, devices that interact with the human body to strengthen or modify the vital field.

2. Prior Art

Previously, U.S. Pat. No. 6,013,021 disclosed an invention that produced an electrical ionization discharge within a magnetic field. Many thousand of that invention, have been sold to the US market and they are much appreciated for their ability to strengthen people's energy in adverse situations like on airplanes and busy shopping malls where people often complain of exhaustion. The magnet was positioned on the back of the neck and the ionizer was placed on the chest. This was somewhat convenient to wear, but somewhat bulky and was not cosmetically attractive. In addition, it was of lower effectiveness because the magnetic field induced at the point of ionization discharge was weak, less than 10 gauss. In addition battery life was limited because 75 mW was required to drive the circuit, limiting battery life to about 50 hours with a relatively large 9 volt battery. In addition the discharge rate was constant creating an undesirable resonance in the vital field, and the housing was made of ABS plastic, which was positioned between the ion discharge and the human body, decreasing effectiveness.

### SUMMARY

In accordance with one embodiment this invention an air gap is ionized across a strong magnetic field at random intervals, strengthening a living body in close proximity to it.

### DRAWINGS—FIGURES

FIG. 1 is a schematic representation of the layout of the invention, showing the components that produce a train of electrical pulses and convert these pulses to high voltage (100V) pulses. It also shows a magnet next to an air gap across which the 100 volt pulses are delivered. Further, it shows the surface of a human body in close proximity to the air gap and magnet, whereby the human body is affected by the electromagnetic interaction.

### DRAWINGS—REFERENCE NUMERALS

10 3V battery
20 Microprocessor
30 FET switching transistor
40 22 uH boost inductor
50 Silicon diode
60 680 uF capacitor
70 Platinum electrodes
80 Air gap
90 NdFe magnet
100 Magnetic field
110 Copper filled housing
120 Human body

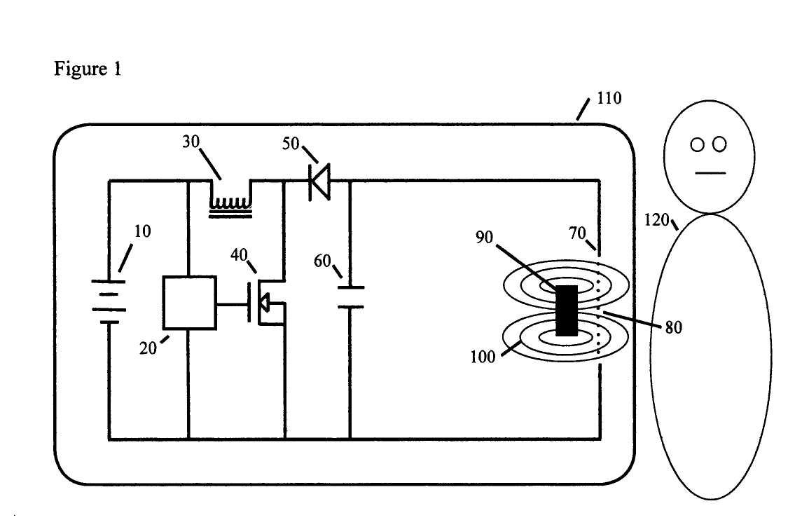

### DETAILED DESCRIPTION OF THE FIRST EMBODIMENT—FIG. 1

3 Volt battery (10) provides power to the electronic circuit including Microprocessor (20) which creates a train of 1 uSec pulses at random intervals between 0.33 and 1 millisecond and applies these pulses to FET switching transistor (30), which, in turn becomes conductive and allows current to flow from 3 v battery (10) through 22 uH boost inductor (40), building up a magnetic field around boost inductor (40). At the end of each pulse, FET switching transistor (30) blocks the current flow through boost inductor (40) which, in turn, reacts with a very high voltage of approximately 100 volts, driving current through silicon diode (50) and into 680 uF capacitor (60). When the magnetic field around boost inductor (40) has completely collapsed, the voltage across boost inductor (40) drops to zero, but silicon diode (50) blocks reverse current flow through it and capacitor (60) remains charged, applying its voltage to platinum wires (70). Between platinum wires (70) is 10 mm air gap (80), comprising electrical field, magnetic field, emitted electrons, and ionized air, which is charged to 100 Volts, creating an electric field strength of 10,000 volts per meter. Platinum electrodes (70) are of such small diameter that a very high voltage gradient develops at their tips, allowing ionization of air gap (80) with less than 200 Volts. Also, magnet (90) is placed within 2 mm of air gap (80), creating a strong magnetic field (100) through air gap (80). Air gap (80), and the entire electronics circuit, are contained within copper filled plastic housing (110). Electrically charged air gap (80) interacts with magnetic field (100) and copper filled housing (110), creating a strengthening influence on human body (120), which is positioned within one inch of air gap (80).

### OPERATION—FIRST EMBODIMENT—FIG. 1

Microprocessor (20) switches a voltage between off and on to create a signal. It also calculates random numbers and scales these numbers to provide a series of random intervals between pulses between 0.33 and 1 millisecond. This is well understood by those familiar with the state of the art. The only uniqueness is that random intervals are applied to a biofield vitalizer. This is valuable because the human body has higher bio-receptiveness to non-linearly varying pulsations as opposed to single frequency pulses.

A train of 100 volt electrical pulses cyclically charges an air gap creating an ionic discharge, while a permanent magnet maintains a strong magnetic field in and around this same air gap such that the electrical and magnetic fields intersect, creating an electromagnetic potential that, through a housing comprising copper and plastic, charges or stimulates a human body. This causes the person to feel more energetic and be more resistant to adverse environmental activity, which might be electromagnetic activity from cell phones or fluorescent lights, or crowds of people with disturbed vital fields (electromagnetic fields related to the nervous and circulatory systems.)

Key components which make this device far more effective than the prior art are:
1. The variable frequency train of pulses results in higher bio-receptiveness than a single frequency pulse train. This can be understood by the analogy that people prefer to listen to music and white noise over a continuous hum or buzz. Not only is a variable frequency pulse train more interesting to the body, but unlike a constant frequency or linearly varying pulse train the body has trouble anticipating and filtering out unpredictable pulses.
2. Copper filled housing (110) comprising about 50% by volume copper resonates with the electromagnetic pulsations and has a greater affinity to human body (120) than a housing made of just plastic, making the use of this invention more enjoyable and more harmonizing than the prior art.
3. The tiny, 0.003 inch diameter, platinum electrodes (70) provide a surprising benefit. They are big enough to conduct the tiny current, but so small that the voltage gradient around the end of the wire is very high, allowing ions in air gap (80) to be emitted and absorbed with much lower voltage. Whereas the prior art uses thousands of volts to create an ion discharge, this invention can achieve the required free ion flow at less than 200 Volts, contributing much to the miniaturization and high efficiency of this invention.
4. Positioning rare earth magnet (90) within 2 mm of ionized air gap (80) causes a very high strength (1700 gauss) magnetic field (compared to 1 to 10 gauss in the prior art) to be present in the air gap as the electrical pulsations pass through it. This very high magnetic field strength allows more powerful vitalizing effects to be produced in human body (120) with far less voltage and current across platinum electrodes (70). In addition to allowing the use of fewer and less expensive components, it allows the use of a single coin cell (the CR2032, 1/10 the size of the nine volt battery in the prior art) to provide 400 hours of operation, (8 times the battery life).
5. Since the entire invention can be made the size of a stack of three quarters, it can easily be placed in the pocket, within 2 inches of the body, within the electrical part of the human biofield surrounding and interpenetrating human body (120).

### CONCLUSIONS RAMIFICATION AND SCOPE

Accordingly the reader will see that, according to one embodiment of the invention, I have provided a biofield vitalizer that is significantly better than the prior art. With variable frequency pulsations and copper filled housing, to increase biocompatibility with a living body, and very high magnetic field strength, and very small diameter electrodes which provide more powerful electromagnetic pulsations at much lower power consumption, this device provides a more effective, more comfortable, and much smaller device with a much longer battery life.

While the above description contains many specificities, these should not be construed as limitations on the scope of any embodiment, but as exemplifications of the presently preferred embodiments thereof. Many other ramifications and variations are possible within the teachings of the various embodiments, For example, The variable electric field might not vary in the form of pulses, but rather continuous signals or bursts of continuous signals. These signals might vary non-linearly. The electrical field might involve air ionization and an ion discharge within an ion discharge chamber as in the current invention, or it might, for example be across a vacuum or other substance, where air ionization might not take place, like a crystal or organic substance. The magnetic field might be modulated or produced by a magnetic coil. The frequency range of pulse train might be anywhere from 1 Hz to megahertz or gigahertz. Additional materials might be added in or close to the air gap to increase effectiveness or bioavailability. As an example, rather than a copper filled plastic housing there might be a piece of natural copper, silver or gold, or copper plated with gold, or other metals or crystals might comprise or be inserted in the housing. Also, this method can be applied to all living bodies, whether plant or animal.

Thus the scope of the invention should be determined by the appended claims and their legal equivalents, and not by the examples given.

1. A method for influencing a living body comprising:
A time varying electrical field stronger than 100 Volts per meter and a magnetic field stronger than 10 gauss; wherein said electrical field intersects said magnetic field, and wherein said intersection of said electric field and said magnetic field is positioned within two inches of a living body.

2. The method described in claim 1 wherein said time varying signal varies non-linearly

3. The method described in claim 1 wherein said time varying signal is a series of pulses

4. The method described in claim 1 wherein a plurality of platinum electrodes of diameter less than 0.006 inch induce said electric field.

5. The method described in claim 1 wherein said electrical field is positioned within a housing comprising copper.

6. The method described in claim 1 wherein said electrical field is applied across an air gap.

7. The method described in claim 1 wherein said electrical field is applied across a material comprising crystal.

8. The method described in claim 1 wherein said influencing is limited to vitalizing.

9. A device comprising an ion emission producing means and an air gap across which said ion emission producing means produces ion emission; wherein said ion emission occurs at non-linearly varying intervals.

10. The method described in claim 9 wherein said ion discharges occur within a magnetic field of field strength exceeding 10 gauss.

11. The method described in claim 9 wherein said ion discharge occurs in an electric field of field strength of less than 20,000 volts per meter.

12. The method described in claim 10 wherein said air gap is positioned within 2 inches of a human body.
